(19) United States
(12) Patent Application Publication
(10) Pub. No.: US 2012/0000460 A1
(43) Pub. Date: Jan. 5, 2012

(54) Method and Device for Nasal Drug Delivery and Nasal Irrigation

(76) Inventor: William J. Flickinger, Lino Lakes, MN (US)

(21) Appl. No.: 12/829,198

(22) Filed: Jul. 1, 2010

(51) Int. Cl. A61M 11/02

(52) U.S. Cl. 128/200.21

(57) ABSTRACT

The present invention incorporates a liquid reservoir incorporated within a main canister that also has an air inlet and at least one air outlet. An insert mates with the air outlet, forming a nozzle that can be inserted into the nasal cavity above the nasal valve. There is a space between the air outlet and the insert. As pressurized air is forced through the air outlet, fluid in the reservoir is drawn up into the space between the insert and air outlet and is atomized in an aerosol mist that is released above the nasal valve independent of the user's breathing. The airstream is sufficient to penetrate the nasal cavity above the inferior turbinate so as to deposit the fluid and provide a washing or irrigation to the upper reaches the nasal cavity.

### TECHNICAL FIELD

The present invention relates to the delivery of fluid to the upper airway in mist or droplet form, either for the irrigation of the nasal passages or the delivery of medication.

### BACKGROUND OF THE INVENTION

Effective delivery of material to the nasal cavity requires a particle size that is large enough to fall out of the airway, delivered under sufficient pressure and airflow to overcome the aerodynamics of the nasal cavity. The nasal cavity is shaped to efficiently deliver air to the lungs. Air enters the nares and passes through the nasal valve, which resides approximately 1.3 cm above the nares and is the narrowest portion of the nose, with a cross-section of at approximately 0.73 cm². The nasal valve is the narrowest anatomic portion of the upper airway, resulting in the volume of air inspired nasally to be efficiently cleansed and humidified by the nasal cavity.

FIG. 1 conceptually illustrates the function of the nasal valve in aerosol delivery that is initiated below the nasal valve. Arrows 120 represent an aerosol flowing into the nasal nares. As illustrated by arrows 121, a portion of this aerosol is reflected off the walls of the nose as the passageway narrows to the nasal valve 130. This reflected material falls out of the nose and is either wasted or is recollected by the device to be delivered repeatedly.

The nasal valve 130 acts to reduce the flow (F) and pressure (P) of that portion of the aerosol stream that crosses the valve and enters the nasal cavity 110. Thus, Flow in (F_{I}) is greater than Flow out (F_{O}), and Pressure in (P_{I}) is greater than Pressure out (P_{O}). As a result, aerosol entering the nasal cavity external to the nasal valve requires a higher pressure and flow rate to achieve the same aerosol distribution as an aerosol introduced internal to the nasal valve.

Air entering the nose meets additional resistance at the level of the inferior turbinate, which directs air downward along the floor of the nose along the least path of resistance. During inhalation, the airflow is dominated by the negative pressure being generated from the lower airway and is directed to the nose from the pharynx. This negative pressure and the structure of the nasal cavity conspire to direct the majority of the air through the lower third of the nose, with very little air entering the upper portion of the nose. Indeed, studies have shown that to reach the upper portion of the nose under the negative pressure of normal breathing, an aerosol must be placed very precisely at the front of the nares. To overcome the aerodynamics of the nose, the delivery system must provide a positive pressure and sufficient airflow to fill the whole nasal cavity.

As we age, we lose the ability to flare the nostrils that we possess as obligate nasal breathers in infancy. The dilator naris muscle becomes less effective at opening the nasal valve as we age, leading to increased problems with effective nasal inspiration in adults, thereby making it more difficult to deliver material from the outside of the nose proximal to the nasal valve.

Prior art methods are designed to deliver particles at the opening of the nares, which may result in significant waste as fluid is reflected off the nasal valve and flows out of the nose, or it may prolong delivery time as the fluid is repeatedly recovered by the delivery system and re-deposited into the nose. Because the nasal valve is the narrowest portion of the nose and is just above the opening of the nares, devices that deliver aerosol below the nasal valve must generate higher pressure and flow rates since the valve acts to lower the pressure and flow as the aerosol passes through it. Prior art methods oftentimes are adaptations of devices designed to deliver fluid to the lower airway and require more interaction from the patient, including long delivery times.

Therefore, it would be desirable to have a method for administering fluid to the upper nasal passage of a patient that requires lower pressure and airflow and produces less mess by virtue of delivery above the nasal valve, and simplicity of use, including short delivery times and normal breathing by the patient.

### SUMMARY OF THE INVENTION

The present invention incorporates a liquid reservoir within a main canister that also has an air inlet and at least one air outlet. An insert mates with the air outlet, creating a space between the air exit portion and the insert portion that is in communication with the reservoir. The insert is fitted with a larger exit than that of the air outlet. As pressurized air is forced through the air exits at an appropriate volume and speed, fluid in the reservoir is drawn up into the space between the insert and air outlet. When the fluid meets the airstream at the exit hole it is atomized into particles conducive to deposition in the upper airway. The airstream is sufficient to penetrate the nasal cavity above the inferior turbinate so as to deposit the fluid and provide a washing or irrigation to the upper reaches the nasal cavity.

The insert and air outlet of the main canister form a nozzle that extends out of the reservoir such that it can be inserted into the nasal cavity so that the mist exits the device approximately at or above the nasal valve. The invention further incorporates an optional cover that is designed to keep the nozzle from contacting the sides of the nose to keep the nozzle from being subjected to pressure that may cause misalignment of the exit holes. The nozzle may alternatively be configured to ensure that it cannot be pushed out of alignment through a series of connections or bonds between the insert and main canister. Furthermore, the inside of the insert is shaped such that a small chamber is formed between the main canister and the insert so that the air is funneled up and out of the exit hole of the insert if misalignment occurs.

In one embodiment, the main canister incorporates feet that enable it to stand up when set on a horizontal surface and may also be designed to fit into a standard docking port of an air compressor to enable the device to remain upright in a hands-free situation so as to be or remained filled with the air supply tube attached.

### BRIEF DESCRIPTION OF THE DRAWINGS

The novel features believed characteristic of the invention are set forth in the appended claims. The invention itself, however, as well as mode of use and advantages thereof, will best be understood by reference to the following detailed description of illustrative embodiments when read in conjunction with the accompanying drawings, wherein:

FIG. 1 conceptually illustrates the function of the nasal valve in aerosol delivery that is initiated below the nasal valve;

FIG. 2 shows an embodiment of a nasal irrigator in accordance with the present invention;

FIG. 3 is a schematic cross section view of the assembled nasal irrigator in accordance with the present invention; and

FIG. 4 shows a perspective view of an assembled nasal irrigator in accordance with the present invention.

### DETAILED DESCRIPTION OF THE DRAWINGS

The pressure and airflow necessary to deliver material to the upper portion of the nose can be reduced if the aerosol is introduced distal of the nares and nasal valve and proximal to the inferior turbinate. The present invention delivers droplets or mists with an air stream and particle sizes designed to stay in the upper airway under sufficient pressure and airflow to overcome the normal aerodynamics of the nose. Unlike prior art methods, the present invention releases mist above the nasal valve, thereby avoid deflection of the fluid off the nasal valve.

Prior art devices that deliver aerosol below the nasal valve must generate higher pressure and flow rates since the valve acts to lower the pressure and flow as the aerosol passes through it. The design of the present invention is directed to the self-administration of fluid to the nasal passages of a patient while ensuring the device fits a wide variety of faces and for simplicity of design, ease of manufacturer. It requires lower pressure and airflow and produces less mess by virtue of delivery above the nasal valve, and simplicity of use, including short delivery times.

The invention delivers fluid to the nasal passages with little interaction required by the user and under sufficient pressure to stent open the airway. The invention delivers particles of a size to ensure that the majority of the mist is retained or deposited within the upper airway, while maximizing the amount of drug delivered and eliminating reflection back from the nasal valve.

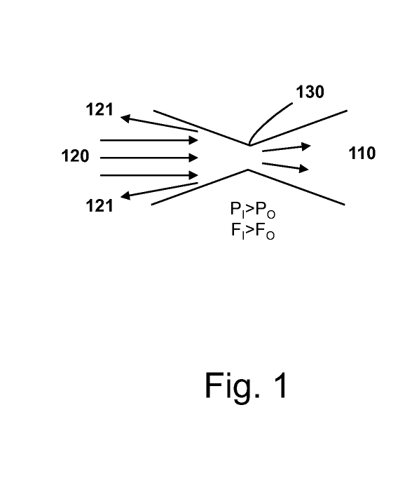

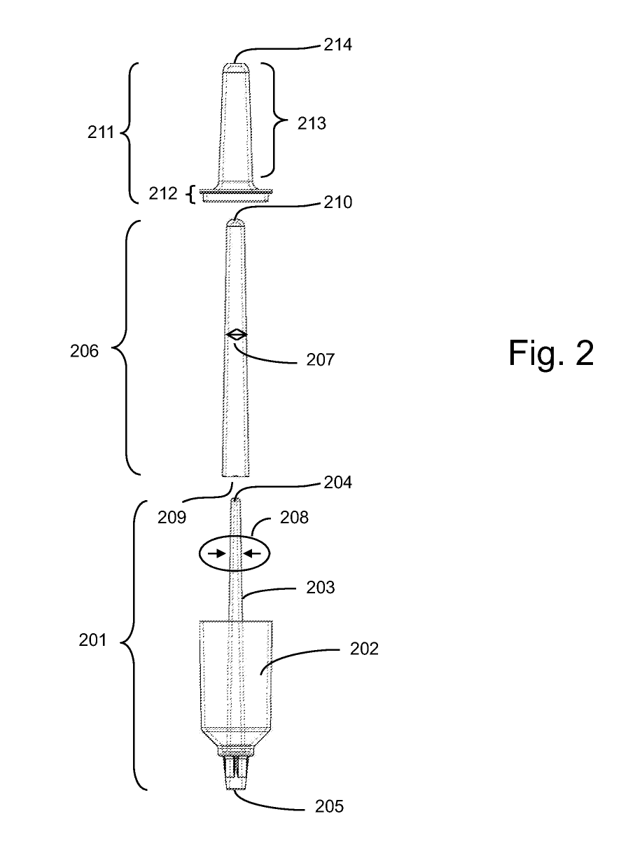

FIG. 2 shows an embodiment of a nasal irrigator in accordance with the present invention. The nasal irrigator comprises three main components. The first component is the main canister 201, which has a fluid reservoir 202 and an air exit port 203 that extends above the reservoir. In one embodiment, the reservoir 202 holds up to 30 ml of fluid or medication. As shown in FIG. 1, the lower portion of the reservoir is downward sloping to ensure fluid collect at the bottom, which allows maximal uptake of fluid through fluid channels (explained below), thereby minimizing waste.

The air exit port 203 has at least one exit hole 204 at the top sufficient to deliver an airstream that is able to atomize fluid and deliver the aerosol to the whole nasal cavity. In one embodiment, the exit hole 204 is between 0.020″ (0.508 mm) and 0.060″(1.524 mm) in diameter and the air exit port has a web-thickness of between 0.030″ (0.762 mm) and 0.060″ (1.524 mm).

The main canister 201 also included an air inlet 205 on the bottom for the admission of pressurized air to create the air stream exiting the air exit port 203.

In one embodiment, the main canister 201 has optional “feet” on the bottom (not shown) for stability. The length of all components on the nozzle cone is limited so that the nozzle cone or its components do not extend past the feet on the main canister when the device is assembled to enable the device to be placed on a flat surface in an upright or standing position. The canister 201 may also be designed to fit into a standard docking port of an air compressor to enable the device to remain upright in a hands-free situation so as to be filled with the air supply tube attached.

The second main component of the nasal irrigator is an insert 206 that fits over the main canister's air exit port 203. The insert 206 can be permanently attached to the canister 201 or it may be removable. The insert 206 has an aerosol exit 210 that is concentrically aligned with the exit hole 204 of the air outlet 203. A peak or extension on the air exit port 203 ensures centering of the insert over the air outlet. The aerosol exit 210 is slightly larger than the exit hole 204 of the air exit port 203 to enable atomization of fluid in the air stream.

The insert 206 has a tapered inner diameter 207 that is larger than and follows the contours of the outer diameter 208 of the air exit port 203. This difference in diameter creates a space of between 0.0001″ (0.00254 mm) and 0.010″ (0.254 mm) between the inner surface of the insert 206 and the outer surface of the air exit port 203. This space allows fluid to be drawn from the reservoir 202 through a channel 209 at the base that is sized to control the fluid flow.

The third main component of the nasal irrigator is the cover 211 that mates with the reservoir 202 of the main canister 201 and extends over the insert 206 such that the insert does not contact the nose as the device is inserted into the nasal cavity, thereby ensuring that the hole 210 in the insert 206 and the hole 204 in the air exit port 203 remain concentrically aligned. The cover 211 includes a mating surface 212 that creates a preferably isodiametric connection to the main canister 201 and extends around the nozzle formed by the insert 206 and air exit port 203. The cover 211 extends just above the insert 206 and has its own exit hole 214 designed not to restrict the flow of the aerosol plume. In one embodiment, the cover 211 provides a cross member or other device that secures the insert 206 to prevent lifting of the insert at the initiation of atomization.

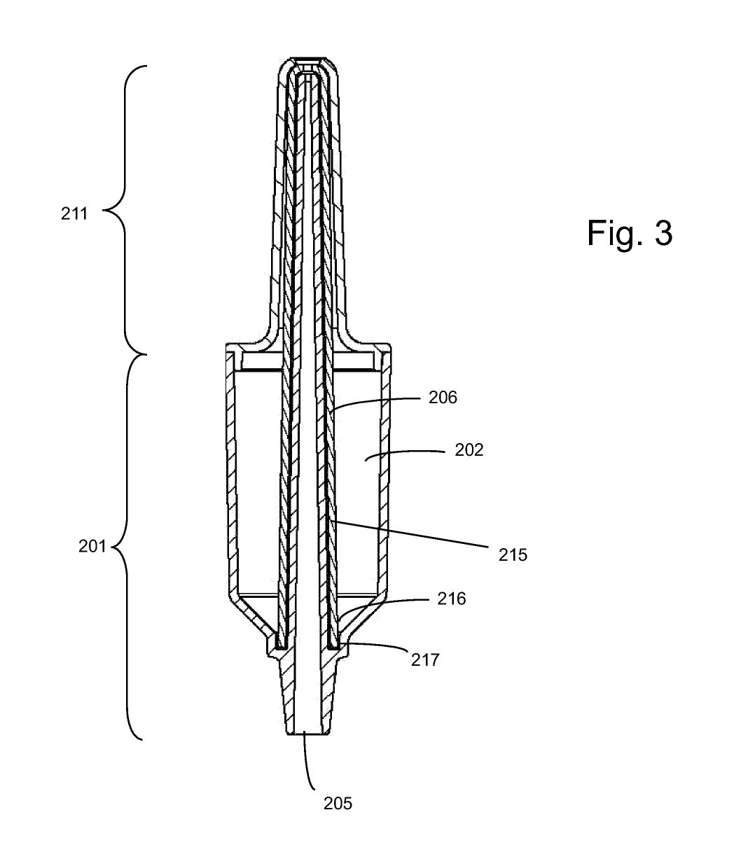

FIG. 3 is a schematic cross section view of the assembled nasal irrigator in accordance with the present invention. This view shows the alignment of the canister 201, insert 206, and cover 211 and the resulting fluid space 215. When fluid is in the reservoir 202 and a pressurized air source is introduced to the system via air inlet 205, a vacuum is created in the space 215 as air exits through outlets 204 and 210. Because the aerosol exit hole 210 in the insert 206 is larger than the exit hole 204 of the air exit port 203, when air is forced through the air exit port 203 at an appropriate volume and speed it creates a venturi effect as the pressurized gas is expelled, thereby drawing fluid in the reservoir 202 up into the space 215 between the insert and air outlet. When the fluid reaches the airstream between the exit holes 204, 210, it is atomized in the airstream to create an aerosol. This aerosol is sufficient to penetrate the nasal cavity above the inferior turbinate so as to the reach the upper nasal cavity.

The aerosol exit 210 in the insert 206 is small enough to ensure that a mist is created yet large enough to ensure that the hole can be chamfered on the outer side to reduce agglomeration of the mist particles upon exit. The aerosol exit hole 210 is chamfered so that the walls of the exit are angled away from a central axis of the hole such that the angle is greater than that of the aerosol plume. This chamfering reduces agglomeration of particles on the walls of the aerosol exit hole 210, resulting in uniformity of particle size across the resultant aerosol plume.

The base 216 of the insert 206 sits in a groove 217 at the base of the canister 201, ensuring that all fluid is scavenged from the bottom of the canister.

The nebulizer components of the present invention can be made from materials such as rigid plastic, glass, metal, ceramic, carbon fiber or other rigid material, an elastomer plastic, or some combination thereof.

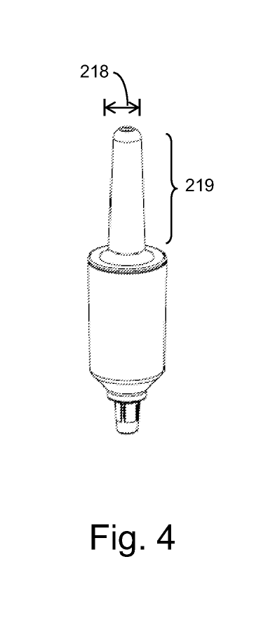

FIG. 4 shows a perspective view of an assembled nasal irrigator in accordance with the present invention. By maintaining a sufficiently narrow nozzle assembly 218, and a sufficiently long and smooth cover 219, the device can be easily and atraumatically inserted into the nose of the patient so that the nozzle 218 extends to or above the nasal valve. The device is then angled by the user to obtain the best distribution based on the user's anatomy. The mist enters the nasal cavity independent of the patient's breathing.

The nasal irrigator of the present invention may also include a feature that guides the user to angle the spray into the nose to a set angle of between 0 and 90 degrees from the vertical plane of the face (defined as the front of the face from the chin to the forehead). For example, one embodiment of the nasal irrigator includes a setoff that sets a specific angle of 30 degrees from the vertical plane of the face. In another embodiment, the setoff angle is 60 degrees from vertical, and in another embodiment the setoff angle is 45 degrees from vertical. The setoff described above is removable to accommodate various size faces and noses.

The method of nasal irrigation of the present invention uses a variable particle size up to 100 microns under a pressure of 1-15 psi (0.069-1.0345 bar), creating a pressurized airflow that enables the resultant air-mist stream to reach the whole nasal cavity independent of the patient's breathing. The resultant aerosol mist reaches the area of the nasal cavity above the inferior nasal turbinate or chonchae to ensure that the mist reaches the areas of the sinus ostia to clear this area of the nasal cavity and enable the natural mucociliary flow to clear the sinuses.

By adjusting the size of the exit holes 204 and 210, the air-fluid mixture can be calibrated to achieve nasal irrigation within a short period of time, without the need for the fluid to exit the nostrils at the time of irrigation, and with a particle size that is designed to loosen the mucous or to enter the sinus cavities, as desired by the end user. In many applications, ideally a mist of 20 microns is delivered at a rate of 0.5 ml per second.

The aerosol mist itself is typically medicated with at least one, and often two or more therapeutic agents. Possible therapeutic agents for use in the medicated mist, either alone or in combination include antibiotics, antifungal agents, corticosteroids and mucolytic agents. The mist may also be medicated with a neurologically active agent targeting the central nervous system through the cranial nerves innervating at least a portion of the nasal cavity.

The description of the present invention has been presented for purposes of illustration and description, and is not intended to be exhaustive or limited to the invention in the form disclosed. Many modifications and variations will be apparent to those of ordinary skill in the art. The embodiment was chosen and described in order to best explain the principles of the invention, the practical application, and to enable others of ordinary skill in the art to understand the invention for various embodiments with various modifications as are suited to the particular use contemplated. It will be understood by one of ordinary skill in the art that numerous variations will be possible to the disclosed embodiments without going outside the scope of the invention as disclosed in the claims.

I claim:

1. A nasal nebulizer, comprising:
(a) a main canister with a reservoir for holding fluid, wherein the canister includes an inlet for pressurized air and at least one air exit port;
(b) an insert that fits within the main canister, wherein the insert fits over the air exit port thereby forming a nozzle, and wherein the insert is larger in diameter than the air exit port, thereby providing a small space between the outer surface of the air exit port and the inner surface of the insert that allows fluid from said reservoir to be drawn upward between the air exit port and insert due to a venturi effect created by pressurized air exiting the air exit port, wherein the fluid is expelled as a mist in an aerosol plume through an aerosol exit hole in the insert; and
(c) a cover that fits over the insert and mates with the main canister and is shaped to be atraumatically inserted into the nasal cavity of a user and extend above the nasal valve, wherein the cover has an exit hole aligned with the aerosol exit hole of the insert and prevents the insert from contacting the inside of the nose when inserted into the nasal cavity, thereby ensuring that the insert remains concentrically aligned with the air exit port.

2. The nebulizer according to claim 1, wherein the air exit port has a least one air exit hole that is 0.020″ (0.508 mm) to 0.060″ (1.524 mm) in diameter.

3. The nebulizer according to claim 1, wherein the air exit port has a web-thickness of 0.030″ (0.762 mm) to 0.060″ (1.524 mm).

4. The nebulizer according to claim 1, wherein the space between the inner surface of the insert and the outer surface of the air exit port is 0.0001″ (0.00254 mm) to 0.010″ (0.254 mm).

5. The nebulizer according to claim 1, wherein the aerosol exit hole in the insert is chamfered so that the walls of the exit are angled away from the central axis of the hole such that the angle is greater than that of the aerosol plume, thereby reducing agglomeration of particles on the walls of the aerosol exit hole, resulting in uniformity of particle size across the aerosol plume.

6. The nebulizer according to claim 1, wherein the main canister has a foot section on the bottom that enables the canister to stand upright when set on a horizontal surface.

7. The nebulizer according to claim 6, wherein said foot section fits into a docking port of an air compressor pump, enabling the nebulizer to remain upright in a hands-free manner.

8. The nebulizer according to claim 1, further comprising a setoff that guides the user to angle the mist into the nose at a set angle of 0-90 degrees from the vertical plane of the face.

9. The nebulizer according to claim 1, wherein the cover includes a mating surface that creates an isodiametric connection to the main canister.

10. A method of nasal irrigation, comprising:
(a) providing fluid in a canister that includes a fluid reservoir, an inlet for pressurized air and at least one air exit port;
(b) mating said air exit port to an insert thereby forming a nozzle, wherein the insert is larger in diameter than the air exit port, thereby providing a space between the outer surface of the air exit port and the inner surface of the insert that allows fluid from said reservoir to be drawn upward between the air exit ports and fluid channels;
(c) atraumatically inserting said nozzle into a user's nose at or above the nasal valve; and
(d) pumping pressurized air through said air exit port, thereby creating a venturi effect that draws fluid from said reservoir upward between the air exit port and insert, expelling the fluid as a mist in an aerosol plume through an aerosol exit hole in the insert and into the user's nasal cavity above the nasal valve independent of the user's breathing, wherein the pressurize air has a pressure of 0.069-1.0345 bar.

11. The method according to claim 10, wherein the aerosol mist has a particle size up to 100 microns.

12. The method according to claim 10, wherein the aerosol mist has a particle size of 20 microns.

13. The method according to claim 10, wherein the aerosol mist is delivered at a rate of 0.5 ml per second.

14. The method according to claim 10, wherein the aerosol mist is medicated with at least one of the follow types of agents:
antibiotic;
antifungal;
corticosteroid;
mucolytic.

15. The method according to claim 10, wherein the aerosol mist is medicated with a neurologically active agent targeting the central nervous system through the cranial nerves innervating at least a portion of the nasal cavity.
